(19) United States
(12) Patent Application Publication
(10) Pub. No.: US 2017/0000429 A1
(43) Pub. Date: Jan. 5, 2017

(54) MOBILE X-RAY APPARATUS

(71) Applicant: HITACHI, LTD., Tokyo (JP)

(72) Inventors: Fumihito NOSE, Tokyo (JP); Kaoru YAMAMOTO, Tokyo (JP)

(73) Assignee: HITACHI, LTD., Tokyo (JP)

(21) Appl. No.: 15/104,250

(22) PCT Filed: Jan. 8, 2015
(86) PCT No.: PCT/JP2015/050305

§ 371 Date: Jun. 14, 2016

(30) Foreign Application Priority Data
Jan. 14, 2014 (JP) 2014-004158

(51) Int. Cl. A61B 6/10; A61B 6/00

(52) U.S. Cl. CPC A61B 6/105; A61B 6/4405; A61B 6/56; B60K 1/04

(57) ABSTRACT

Provided is a mobile X-ray apparatus which can be manually moved even in a case where a battery is exhausted. The mobile X-ray apparatus according to the present invention is configured to include a motor that drives a driving wheel of a mobile cart via a clutch, a battery that supplies electric power to the motor, a cart manipulation unit that manipulates movement of the mobile cart, and a selector switch that switches between two manipulation states which are a normal manipulation state and a manual manipulation state of the mobile cart. The motor is configured to brake when electric power stops being supplied from the battery. In a case where the manual manipulation state is selected, electric power stops being supplied from the battery to the motor, and the driving wheel and the motor can be controlled so as to be attached to and be detached from each other.

### TECHNICAL FIELD

The present invention relates to a mobile X-ray apparatus which can be moved by a mobile cart.

### BACKGROUND ART

As an X-ray device which irradiates an object with an X-ray, there is a mobile X-ray apparatus which can be moved by a mobile cart. The mobile X-ray apparatus has a built-in battery, and the battery realizes operation of the X-irradiation device and driving of a motor.

As such a mobile X-ray apparatus, there has been a method of reducing the possibility of an exhausted battery by determining whether or not the battery is charged based on a residual quantity of the battery (PTL 1).

### CITATION LIST

### Patent Literature

PTL 1: JP-A-2012-095715

### SUMMARY OF INVENTION

### Technical Problem

PTL 1 discloses a mobile X-ray apparatus in which a battery is unlikely to be exhausted. However, there is no disclosure regarding a case where the battery is exhausted.

For example, in a motor which is adopted to such a mobile X-ray apparatus, the motor itself has a built-in brake, and the brake is applied except when being energized. In a case where such a motor is used, when the battery is exhausted, not only can the motor not be driven, but also the brake cannot be released. Accordingly, the mobile X-ray apparatus cannot be moved.

The present invention has been made in consideration of the aforementioned problem, and an object thereof is to provide a mobile X-ray apparatus which can be manually moved even in a case where a battery is exhausted.

### Solution to Problem

In order to achieve the aforementioned object, a mobile X-ray apparatus according to the present invention is configured to include a mobile cart, an X-ray device that is mounted in the mobile cart and irradiates an object with an X-ray, a motor that drives a driving wheel of the mobile cart via a clutch, a battery that supplies electric power to the motor, a cart manipulation unit that manipulates movement of the mobile cart, and a selector switch that switches between two manipulation states which are a normal manipulation state and a manual manipulation state of the mobile cart. The motor is configured to brake when electric power stops being supplied from the battery. In a case where the normal manipulation state is selected by the selector switch, supplying of electric power to the motor is controlled in response to a manipulation of the cart manipulation unit. In a case where the manual manipulation state is selected by the selector switch, electric power stops being supplied from the battery to the motor, and the clutch is manipulated via a manipulation transmission mechanism in response to a manipulation of the cart manipulation unit. The driving wheel and the motor are controlled so as to be attached to and be detached from each other in response to a manipulation of the clutch.

According to such a configuration, in a case where the battery is exhausted, it is possible to cause the apparatus to be movable by switching over to the manual manipulation. In this case, a brake of the motor may be used through a manipulation of the clutch.

### Advantageous Effects of Invention

According to the present invention, it is possible to provide the mobile X-ray apparatus which can be manually moved even in a case where the battery is exhausted.

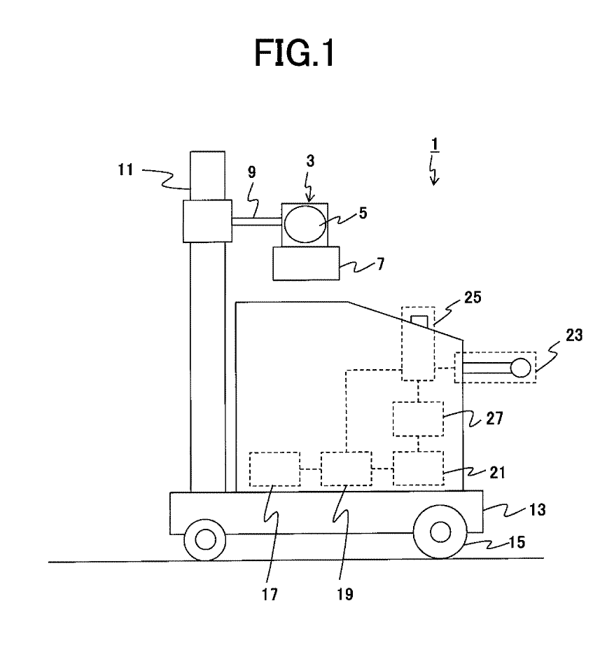

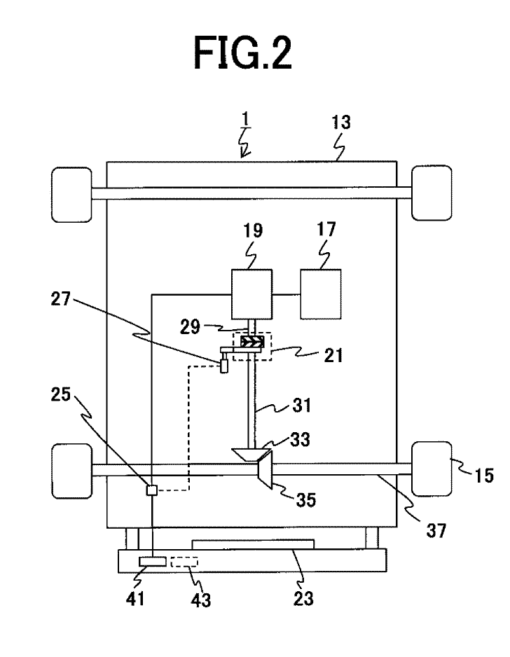

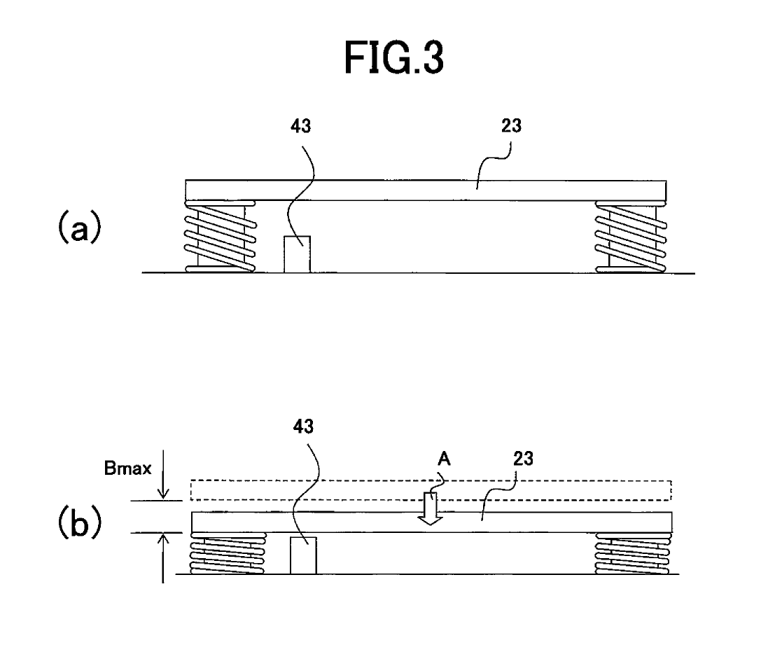

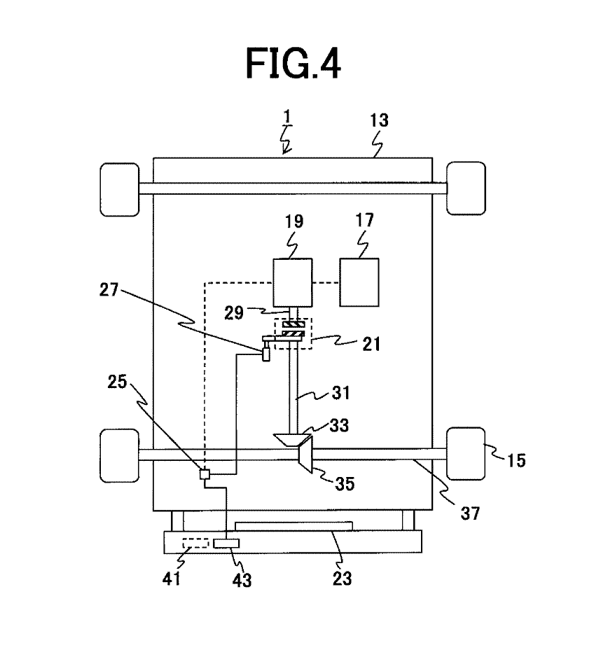

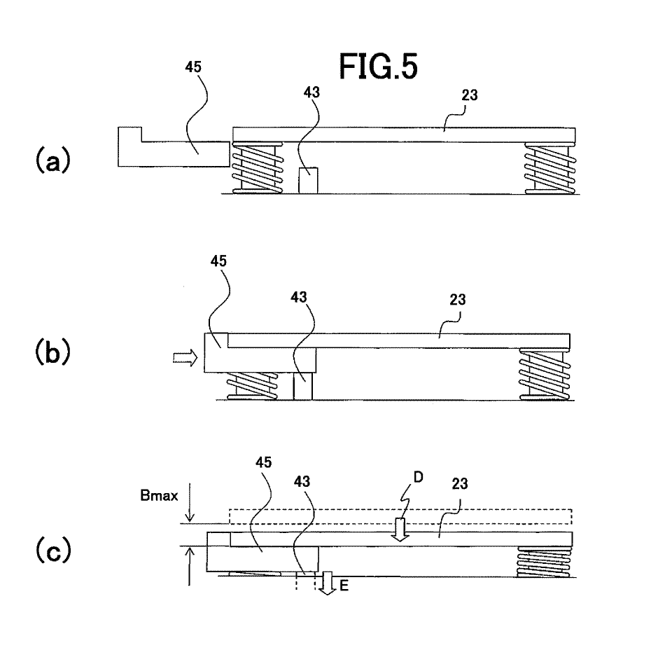

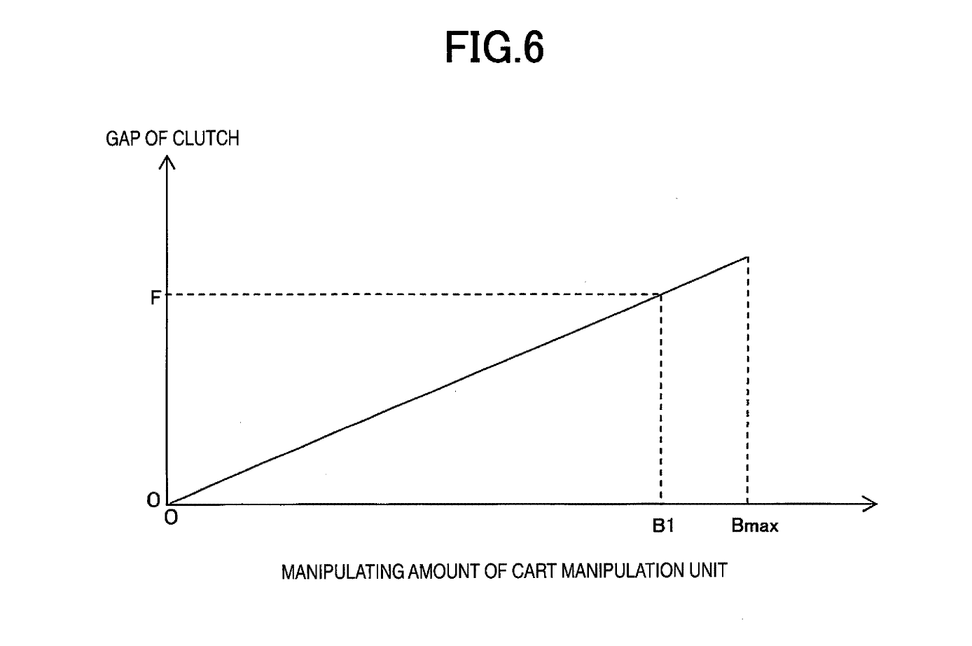

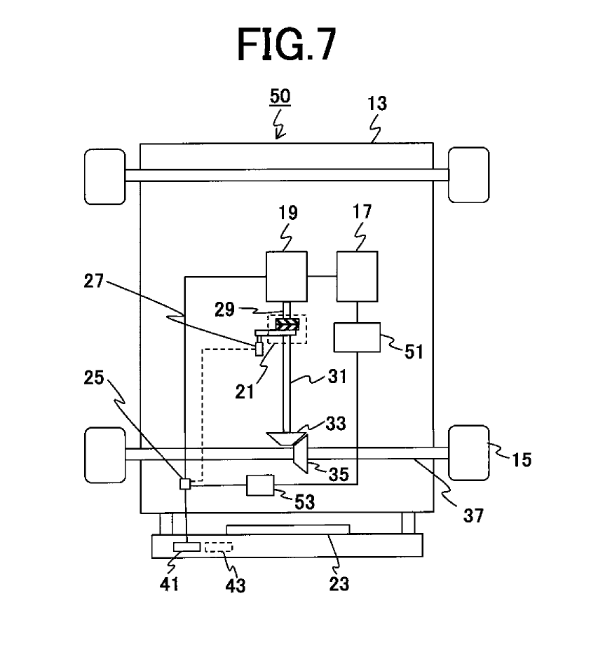

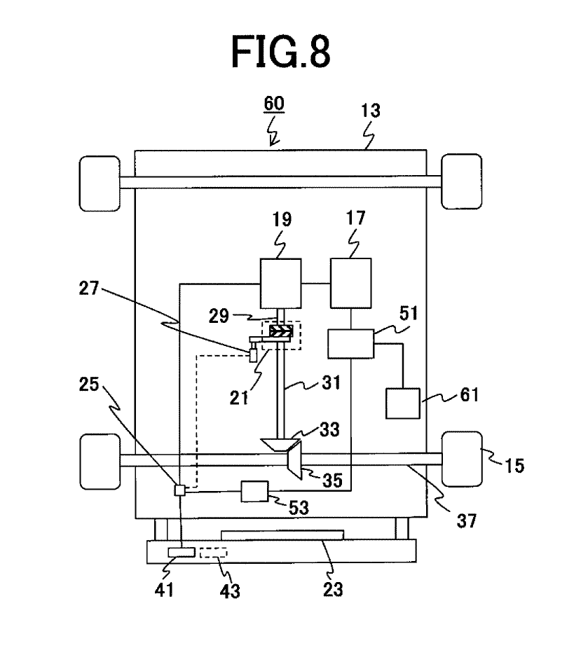

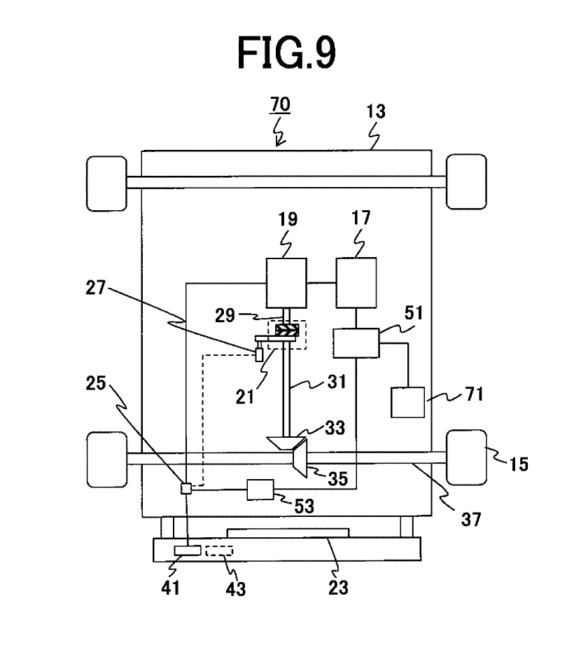

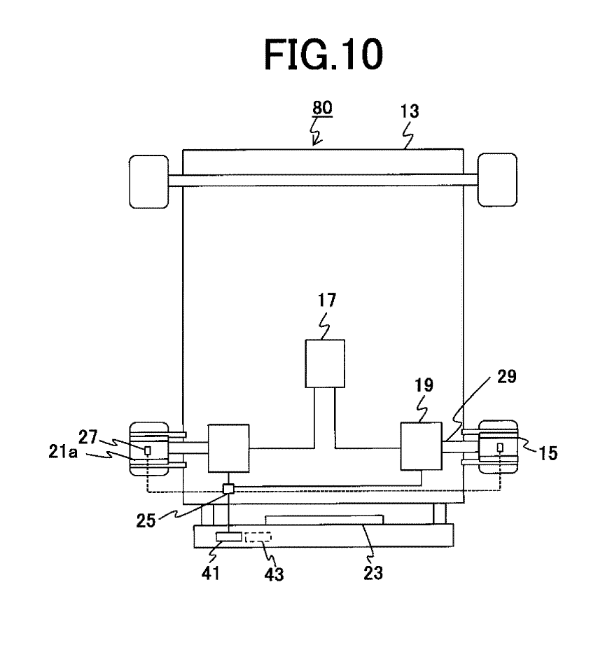

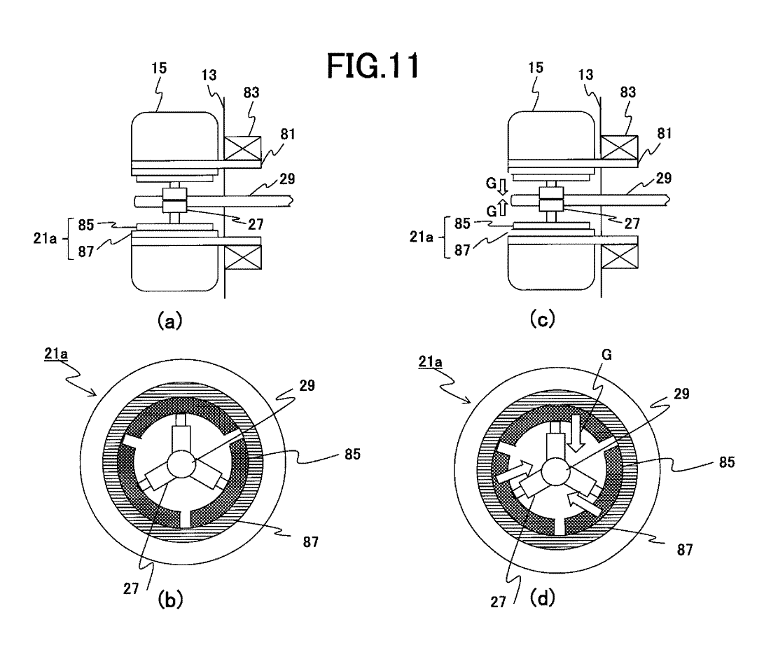

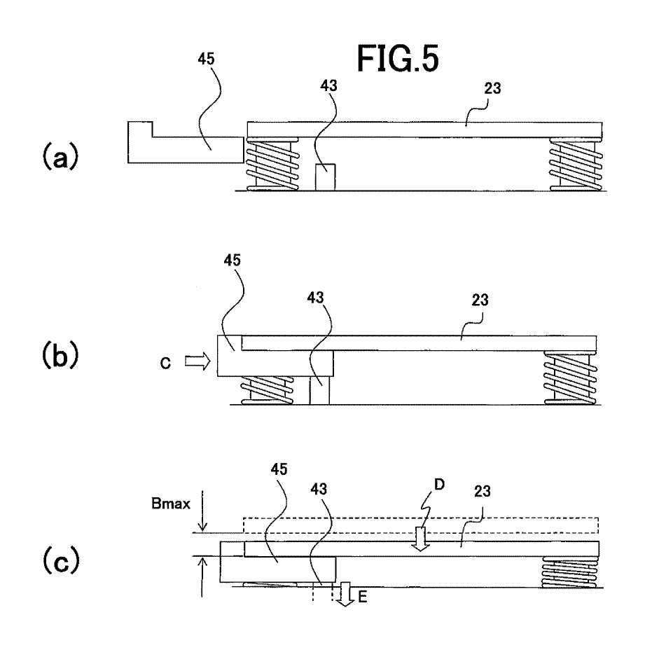

### BRIEF DESCRIPTION OF DRAWINGS

[FIG. 1] FIG. 1 is a configuration diagram illustrating a mobile X-ray apparatus 1.

[FIG. 2] FIG. 2 is another configuration diagram illustrating the mobile X-ray apparatus 1.

[FIG. 3] FIG. 3(a) is a schematic view illustrating a state before a cart manipulation unit 23 is manipulated, and FIG. 3(b) is a schematic view illustrating a state after the cart manipulation unit 23 is manipulated.

[FIG. 4] FIG. 4 is a configuration diagram illustrating the mobile X-ray apparatus 1 in a state where the cart manipulation unit 23 is manipulated in a manual manipulation state.

[FIG. 5] FIG. 5(a) is a schematic view illustrating a state before a spacer 45 is inserted at the time of the manual manipulation state, FIG. 5(b) is a schematic view illustrating a state after the spacer 45 is inserted, and FIG. 5(c) is a schematic view illustrating a state after the cart manipulation unit 23 is manipulated in addition thereto.

[FIG. 6] FIG. 6 is a diagram illustrating a relationship between a manipulating amount of a cart manipulation unit and a clutch space.

[FIG. 7] FIG. 7 is a configuration diagram illustrating a mobile X-ray apparatus 50.

[FIG. 8] FIG. 8 is a configuration diagram illustrating a mobile X-ray apparatus 60.

[FIG. 9] FIG. 9 is a configuration diagram illustrating a mobile X-ray apparatus 70.

[FIG. 10] FIG. 10 is a configuration diagram illustrating a mobile X-ray apparatus 80.

[FIG. 11] FIG. 11(a) is a schematic sectional view illustrating a state where a clutch 21a is engaged, FIG. 11(b) is a schematic view illustrating the clutch 21a in an engaged state, FIG. 11(c) is a schematic sectional view illustrating a state where the clutch 21a is disengaged, and FIG. 11(d) is a schematic view illustrating the clutch 21a in a disengaged state.

### DESCRIPTION OF EMBODIMENTS

A mobile X-ray apparatus according to the present embodiment includes a mobile cart, an X-ray device that is mounted in the mobile cart and irradiates an object with an X-ray, a motor that drives a driving wheel of the mobile cart via a clutch, a battery that supplies electric power to the motor, a cart manipulation unit that manipulates movement of the mobile cart, and a selector switch that switches between two manipulation states which are a normal manipulation state and a manual manipulation state of the mobile cart. The motor is configured to brake when electric power stops being supplied from the battery. In a case where the normal manipulation state is selected by the selector switch, supplying of electric power to the motor is controlled in response to a manipulation of the cart manipulation unit. In a case where the manual manipulation state is selected by the selector switch, electric power stops being supplied from the battery to the motor, and the clutch is manipulated via a manipulation transmission mechanism in response to a manipulation of the cart manipulation unit. The driving wheel and the motor are controlled so as to be attached to and be detached from each other in response to a manipulation of the clutch.

In addition, the manipulation transmission mechanism includes a manipulation switch, a spacer which can perform inserting and pulling-out operations between the manipulation switch and the cart manipulation unit, and a clutch operation unit which operates the clutch in accordance with a push-in amount when the manipulation switch is pushed in. The manipulation switch is configured to be pushed in via the spacer which is inserted between the cart manipulation unit and the manipulation switch, in response to a manipulation of the cart manipulation unit.

In addition, the manipulation transmission mechanism is configured to cause a maximum manipulating amount in a case where the cart manipulation unit is manipulated when the spacer is inserted to be substantially equal to a maximum manipulating amount in a case where the cart manipulation unit is manipulated when the spacer is not inserted.

In addition, a voltage detection unit that detects a residual quantity of an electric capacity of the battery, and a switch operation unit that operates the selector switch are further included. When the residual quantity of the electric capacity detected by the voltage detection unit becomes equal to or less than a predetermined value, the switch operation unit operates the selector switch so as to switch over to the manual manipulation state from the normal manipulation state.

In addition, the battery also supplies electric power to the X-ray device and further includes a storage unit which stores an estimated number of usages of the X-ray device set in advance. The switch operation unit operates the selector switch so as to cause the residual quantity of the electric capacity detected by the voltage detection unit to ensure at least an electric capacity allowing as many times of irradiation with an X-ray as the estimated number of usages.

In addition, in a case where it is determined that the residual quantity of the electric capacity detected by the voltage detection unit while the mobile cart is moving in the normal manipulation state becomes equal to or less than a predetermined value, the switch operation unit stops the motor and operates the selector switch after the mobile cart stops.

In addition, an alarm unit that issues a warning to a user in a case where the voltage detection unit determines that the residual quantity of the electric capacity of the battery is equal to or less than a warning residual quantity is further included. When the residual quantity of the electric capacity detected by the voltage detection unit becomes equal to or less than a predetermined value which is smaller than the warning residual quantity, the switch operation unit operates the selector switch.

Hereinafter, the mobile X-ray apparatus according to the present invention will be described in detail.

### Embodiment 1

Hereinafter, with reference to the drawings, the embodiment of the present invention will be described. FIG. 1 is a schematic side view illustrating a mobile X-ray apparatus 1. The mobile X-ray apparatus 1 is mainly configured to include an X-ray device 3, a mobile cart 13, a battery 17, a motor 19, a clutch 21, a cart manipulation unit 23, a selector switch 25, a clutch operation unit 27, and the like.

The X-ray device 3 includes an X-ray generation unit 5, an X-ray movable collimator 7, and the like. The X-ray generation unit 5 is a portion which generates an X-ray in response to a manipulation of a manipulation panel (not illustrated). In addition, the X-ray movable collimator 7 is a portion which adjusts an irradiation field of an X-ray. The X-ray movable collimator 7 is rotatably mounted immediately under the X-ray generation unit 5.

The X-ray device 3 is fixed to an arm 9. The arm 9 is attached to a strut 11 which stands in the front of the mobile cart 13. The strut 11 is rotatable with respect to the mobile cart 13. Therefore, the arm 9 can turn to the front of the mobile X-ray apparatus 1 (to the left side in the diagram). In addition, the arm 9 can move in the vertical direction with respect to the strut 11. Moreover, the arm 9 is extendable.

In a case where the X-ray device 3 is used, first, the mobile X-ray apparatus 1 is moved to a place in the vicinity of the object. Subsequently, the strut 11 is rotated, and the X-ray device 3 is moved to a position above the object, thereby adjusting the position by raising, lowering, or extending the arm 9. An X-ray detector is disposed under the object in advance. In other words, the X-ray detector is disposed so as to face the X-ray generation unit 5. For example, the X-ray detector is a flat panel detector (FPD).

In this state, when the X-ray device irradiates the object with an X-ray, the X-ray which has been transmitted through the object is detected by the X-ray detector . The X-ray detector generates an electrical signal corresponding to the transmitted X-ray. As described above, an X-ray image of the object can be captured. The X-ray detector may be a film or an imaging plate which accumulates a signal of the transmitted X-ray as a latent image.

The battery 17 and the motor 19 are mounted in the mobile cart 13. The battery 17 operates the X-ray device 3 and drives the motor 19. The batteries can be respectively mounted in the X-ray device 3 and the motor 19. The motor 19 is a deadman type in which a brake is applied except when being energized.

The selector switch 25 switches between the manipulation states of the mobile cart 13. In other words, switching can be made between movement by the motor 19 and movement by the manual manipulation. Each of the manipulation states will be described later in detail.

The cart manipulation unit 23 is a portion which manipulates movement of the mobile cart 13. In the normal manipulation state (in a state where the mobile cart 13 is moved by the battery 17), electricity is supplied to the motor 19 from the battery 17. The brake inside the motor 19 can be manipulated by manipulating the cart manipulation unit 23. Therefore, the motor 19 can be driven by manipulating the cart manipulation unit 23.

FIG. 2 is a schematic plan view illustrating the structure of the mobile cart 13. FIG. 2 is a diagram illustrating the normal manipulation state. A motor shaft 29 of the motor 19 is connected to a propeller shaft 31 via the clutch 21. In other words, in a state where the clutch 21 is engaged, the propeller shaft 31 is rotated by the motor 19. Rotary force of the propeller shaft 31 is transmitted to a shaft 37 via gears 33 and 35. A driving wheel 15 is fixed to the shaft 37. In other words, when the rotary force from the propeller shaft 31 is transmitted to the shaft 37, the driving wheel 15 can be driven. Therefore, the mobile cart 13 can travel.

The clutch operation unit 27 is connected to the clutch 21. The clutch operation unit 27 is a portion performing engagement and disengagement between a clutch plate and a flywheel which the clutch 21 is configured to include. In the normal manipulation state, the clutch 21 is maintained in the engaged state at all times. In other words, the driving force of the motor 19 is surely transmitted to the shaft 37 via the propeller shaft 31 and the gears 33 and 35.

A manipulating amount detection unit 41 is provided in the vicinity of the cart manipulation unit 23. The manipulating amount detection unit 41 detects a manipulating amount of the cart manipulation unit 23. In addition, a manipulation switch 43 is provided in the vicinity of the cart manipulation unit 23. The manipulation switch 43 is a portion which operates the clutch operation unit 27. The manipulation switch 43 and the clutch operation unit 27 do not use electric power and are configured to be hydraulic circuits or mechanical mechanisms, for example.

Subsequently, a method of manipulating the mobile X-ray apparatus 1 will be described. FIG. 3(a) is a schematic view of the cart manipulation unit 23. For example, the cart manipulation unit 23 is a manipulation lever. When being released from the grasp, the cart manipulation unit 23 returns to the original state due to an elastic member. As illustrated in FIG. 3(b), when the cart manipulation unit 23 is manipulated (the arrow A in the diagram), the manipulating amount is detected by the manipulating amount detection unit 41. In other words, the displacement magnitude of the cart manipulation unit 23 is detected. For example, the manipulating amount detection unit 41 is configured to be a position sensor or a slide resistor.

The manipulating amount detection unit 41 drives the motor 19 in accordance with the manipulating amount of the cart manipulation unit 23. For example, in a case where the manipulating amount of the cart manipulation unit 23 is small, the manipulating amount detection unit 41 drops the rotation frequency of the motor 19. As the manipulating amount of the cart manipulation unit 23 increases, the rotation frequency of the motor 19 is controlled so as to rise. Therefore, a user can adjust the moving speed of the mobile cart 13 in accordance with the manipulating amount of the cart manipulation unit 23.

As described above, in a case where the manipulating amount of the cart manipulation unit 23 is zero (the state in FIG. 3(a)), the motor 19 stops the propeller shaft 31 from rotating by using the brake of the motor 19 itself. Therefore, the driving wheel 15 does not rotate. In this manner, in a state of being released from the grasp of the user, the driving wheel 15 is in a state where the brake is applied at all times .

In addition, the maximum value of the manipulating amount of the cart manipulation unit 23 is mechanically limited. As illustrated in FIG. 3(b), the maximum manipulating amount of the cart manipulation unit 23 is referred to as Bmax. The cart manipulation unit 23 cannot be manipulated at the displacement magnitude equal to or greater than the Bmax. Therefore, in a case where the manipulating amount of the cart manipulation unit 23 is the Bmax, the mobile cart 13 travels at the maximum speed.

Here, the manipulation switch 43 is provided in the manipulation direction of the cart manipulation unit 23. However, in the normal manipulation state, even though the cart manipulation unit 23 is manipulated at the maximum amount (Bmax), the cart manipulation unit 23 and the manipulation switch 43 do not come into contact with each other. In other words, even though the cart manipulation unit 23 is manipulated, the manipulation switch 43 is not manipulated.

Subsequently, a case where the battery 17 is exhausted (a case where the residual quantity thereof becomes equal to or less than a predetermined quantity) will be described. FIG. 4 is a schematic plan view illustrating the operative structure of the mobile cart 13 in the manual manipulation state. When the battery 17 is exhausted, the motor 19 can no longer be driven. In such a case, first, switching of the selector switch 25 is performed. The manual manipulation state can be realized by performing switching of the selector switch 25.

When switching of the selector switch 25 is performed, supplying of electric power to the motor 19 is blocked. Therefore, even though the cart manipulation unit 23 is manipulated, the motor 19 does not operate. In other words, the motor 19 is in a state where the brake is in operation at all times. In this state, the clutch operation unit 27 can be operated by the manipulation switch 43. The manipulation switch 43 is manipulated as follows.

First, as illustrated in FIG. 5(a), a spacer 45 is disposed in the vicinity of the cart manipulation unit 23 in the normal manipulation state. When the selector switch 25 is manipulated, as illustrated in FIG. 5(b), the spacer 45 is automatically or manually inserted into a gap between the cart manipulation unit 23 and the manipulation switch 43 (the arrow C in the diagram).

In this state, when the cart manipulation unit 23 is manipulated (the arrow D in the diagram), in accordance with the manipulating amount of the cart manipulation unit 23, the manipulation switch 43 is pushed in (the arrow E in the diagram). In other words, the manipulation switch 43 can be manipulated by the cart manipulation unit 23. As long as the manipulation switch 43 can be manipulated, the spacer 45 is not necessarily essential.

Here, it is desirable that the maximum value of the manipulating amount of the manipulation switch 43 is substantially the same as the maximum value (Bmax) of the manipulating amount of the cart manipulation unit 23 in the normal manipulation state. In other words, the gap between the cart manipulation unit 23 and the manipulation switch 43 and the thickness of the spacer 45 substantially coincide with each other. Consequently, the maximum push-in amount of the manipulation switch 43 via the spacer 45 becomes substantially equal to the maximum manipulating amount (Bmax) of the cart manipulation unit 23 in the normal manipulation state. Therefore, there is no difference for the user in the sense of manipulation between the normal manipulation state and the manual manipulation state.

When the manipulation switch 43 is pushed in, the clutch operation unit 27 operates in accordance with the push-in amount of the manipulation switch 43. The clutch 21 which is in the engaged state at all times while being normally manipulated can be disengaged by the clutch operation unit 27. For example, in the normal manipulation state where the clutch plate and the flywheel are pressed together by the elastic member, the gap can be provided between the flywheel and the clutch plate by using a hydraulic actuator or the like and moving the clutch plate.

In this manner, in the manual manipulation state, the clutch 21 is manipulated via the manipulation transmission mechanism by manipulating the cart manipulation unit 23. In other words, in the above-described example, the manipulation transmission mechanism is configured to include the manipulation switch 43, the spacer 45 which can perform the inserting and pulling-out operations between the manipulation switch 43 and the cart manipulation unit 23, and the clutch operation unit 27 which operates the clutch 21 in accordance with the push-in amount when the manipulation switch 43 is pushed in.

The manipulation transmission mechanism is not limited to the above-described example. For example, an operation mechanism of the clutch operation unit 27 with respect to the manipulation switch 43 is not necessarily hydraulic and may have a different mechanical link structure. However, it is desirable that the mechanism does not use electric power.

FIG. 6 is a conceptual diagram illustrating a relationship of the gap of the clutch (the gap between the clutch plate and the flywheel. The same applies hereinafter) with respect to the manipulating amount of the cart manipulation unit 23. As illustrated, the manipulating amount of the cart manipulation unit 23 is substantially proportional to the gap of the clutch. When the manipulating amount of the cart manipulation unit 23 is increased, the gap of the clutch increases. In other words, the clutch plate and the flywheel are likely to slip.

Here, in a case where the manipulating amount of the cart manipulation unit 23 is zero, the gap of the clutch becomes zero. In other words, a state similar to the normal manipulation state is realized. Therefore, the shaft 37 stops from moving due to braking of the motor 19. Accordingly, in a case where the manipulating amount of the cart manipulation unit 23 is zero, similar to the normal manipulation state, the mobile cart 13 does not move due to braking of the motor 19.

When the cart manipulation unit 23 is manipulated and the manipulating amount is increased, the gap of the clutch gradually increases. Therefore, slipperiness is generated between the clutch plate and the flywheel. Accordingly, the mobile cart 13 can be moved. In addition, as the manipulating amount is increased, the gap of the clutch increases, and slipperiness between the clutch plate and the flywheel also increases. Therefore, moving resistance of the mobile cart 13 becomes small. Accordingly, the mobile cart 13 can be easily moved.

In FIG. 6, when the manipulating amount of the cart manipulation unit 23 becomes B1, the clutch plate and the flywheel are completely disengaged from each other. In other words, the mobile cart 13 can be moved without being influenced by braking of the motor 19.

The maximum value Bmax of the manipulating amount may be caused to approximately coincide with B1. However, it is desirable that B1 is set to the extent of substantially 70% to 80% of the Bmax. When B1 is excessively small with respect to the Bmax, it is difficult to perform fine adjustment of the brake for the change of the manipulating amount. In addition, when B1 is excessively close to the Bmax, the brake is in operation while only slightly loosening the cart manipulation unit 23. Therefore, unless the cart manipulation unit 23 is manipulated at the maximum amount at all times, the mobile cart 13 is not in a free state (a state of not being influenced by braking of the motor 19), and a great load is applied to the user. Therefore, it is desirable that B1 is set to the extent of substantially 70% to 80% of the Bmax.

Hereinbefore, in the mobile X-ray apparatus 1 according to the present embodiment, even in a case where the battery 17 is exhausted, the mobile X-ray apparatus 1 can be manually moved by manipulating the mobile cart 13. In addition, in this case, since the brake of the motor 19 is utilized, the movement is safe.

In addition, both the normal manipulation state and the manual manipulation state can be manipulated by the same cart manipulation unit 23, and the manipulation methods thereof are the same as each other. Moreover, the manipulating amounts of the cart manipulation unit 23 in the normal manipulation state and the manual manipulation state are substantially the same as each other. Therefore, the user can manipulate the mobile cart 13 without feeling a sense of incompatibility even at the time of the manual manipulation state.

In this case, in the manual manipulation state, since the manipulating amount of the cart manipulation unit 23 and the gap of the clutch are in a proportional relationship, the brake can be gradually applied in accordance with the manipulating amount of the cart manipulation unit 23. Therefore, the brake can be prevented from being suddenly applied. In addition, there is no need to separately provide another brake for manual manipulation.

### Embodiment 2

Subsequently, another embodiment will be described. FIG. 7 is a diagram illustrating a mobile X-ray apparatus 50. In the description below, the same reference signs in FIGS. 1 to 6 will be applied to the configurations which exhibit functions similar to those in the mobile X-ray apparatus 1, and description thereof will be omitted.

The mobile X-ray apparatus 50 has a configuration approximately similar to that of the mobile X-ray apparatus 1. However, the mobile X-ray apparatus 50 is different therefrom in the point that a voltage detection unit 51 and a switch operation unit 53 are provided. The voltage detection unit 51 detects a voltage of the battery 17. Therefore, the residual quantity of electric power of the battery 17 can be detected by the voltage detection unit 51.

When the voltage detection unit 51 detects that the residual quantity of electric power of the battery 17 is equal to or less than a predetermined quantity, the switch operation unit 53 connected to the voltage detection unit 51 performs switching of the selector switch 25. In other words, when the residual quantity of the battery 17 becomes equal to or less than a predetermined quantity, switching over to the manual manipulation state from the normal manipulation state is automatically performed.

There are cases where the voltage detection unit 51 detects a decrease of the residual quantity of the battery 17 while the mobile X-ray apparatus is travelling by the motor 19. However, switching of the selector switch 25 automatically performed during travelling is dangerous. Therefore, before performing switching of the selector switch 25, first, the switch operation unit 53 gradually stops electric power from being supplied from the battery 17 to the motor 19. The mobile cart 13 is caused to gradually stop first. After the motor 19 completely stops, the switch operation unit 53 performs switching of the selector switch 25.

Moreover, after switching of the selector switch 25 is performed, the switch operation unit 53 may move the spacer 45 and may insert the spacer 45 between the cart manipulation unit 23 and the manipulation switch 43.

According to a second embodiment, it is possible to obtain an effect similar to that of a first embodiment. In addition, when the residual quantity of the battery decreases due to the voltage detection unit 51, switching over to the manual manipulation state can be automatically performed.

### Third Embodiment

Subsequently, a third embodiment will be described. FIG. 8 is a diagram illustrating a mobile X-ray apparatus 60. The mobile X-ray apparatus 60 has a configuration approximately similar to that of the mobile X-ray apparatus 50. However, the mobile X-ray apparatus 60 is different therefrom in the point that a storage unit 61 is provided. The storage unit 61 stores estimated usage of the X-ray device 3. In the mobile X-ray apparatus 60, electric power is supplied from one battery 17 to both the X-ray device 3 and the motor 19.

In the mobile X-ray apparatus 60, the estimated usage of a current day is stored in the storage unit 61. For example, the number of times of irradiation with an X-ray is stored in the storage unit 61. In addition, the storage unit 61 stores a usage quantity of electric power required for performing irradiation once with an X-ray.

The voltage detection unit 51 calculates the minimum residual quantity of electric power required for performing irradiation with an X-ray, based on the estimated number of usages of an X-ray and the usage quantity of electric power per time. After the X-ray device 3 is used (after irradiation is performed with an X-ray), the number of usages is subtracted from the estimated number of usages, and the residual quantity of electric power necessary for the remaining number of times of irradiation with an X-ray is calculated every time. Such calculation of the required minimum residual quantity of electric power may be performed by adopting a separate control unit.

As described above, the voltage detection unit 51 detects the residual quantity of electric power of the battery 17. When the voltage detection unit 51 detects that the residual quantity of electric power of the battery 17 is the minimum residual quantity of electric power ensuring the remaining number of times of irradiation with an X-ray, the switch operation unit 53 performs switching of the selector switch 25. During travelling, as described above, after the motor 19 is caused to stop, switching of the selector switch 25 is performed.

According to the third embodiment, it is possible to obtain an effect similar to that of the second embodiment. In addition, an electric capacity allowing as many times of irradiation with an X-ray as the estimated number of times can be ensured by the voltage detection unit 51. Therefore, it is possible to prevent irradiation with an X-ray from being impossible due to insufficient battery capacity, after the mobile X-ray apparatus 60 is moved.

### Fourth Embodiment

Subsequently, a fourth embodiment will be described. FIG. 9 is a diagram illustrating a mobile X-ray apparatus 70. The mobile X-ray apparatus 70 has a configuration approximately similar to that of the mobile X-ray apparatus 50. However, the mobile X-ray apparatus 70 is different therefrom in the point that an alarm unit 71 is provided.

When a residual quantity of the battery becomes equal to or less than a predetermined quantity (equal to or less than a warning residual quantity) due to the voltage detection unit 51, first, the alarm unit 71 operates. The alarm unit 71 warns the user of the decreased residual quantity of the battery through a sound, light, and others.

In this state, when the battery 17 is additionally used, and the residual quantity of the battery falls further below a predetermined quantity, the switch operation unit 53 performs switching of the selector switch 25. The residual quantity of the battery at which switching over to the manual manipulation state is performed may be the minimum quantity of electric power required for driving the motor 19. Otherwise, as described above, the residual quantity thereof may be the minimum quantity of electric power required for performing the remaining irradiation with an X-ray while the storage unit is separately provided. Any one thereof is acceptable as long as the user can recognize that the residual quantity of the battery has decreased, through the alarm unit 71 before switching over to the manual manipulation state is performed.

According to the fourth embodiment, it is possible to obtain an effect similar to that of the second embodiment . In addition, when the residual quantity of the battery 17 decreases, switching over to the manual manipulation state is not performed unexpectedly. An alarm of switching over to the manual manipulation is issued beforehand. Therefore, the user can pay attention to exhaustion of the battery while refraining from unnecessarily manipulating the cart, and the like.

### Fifth Embodiment

Subsequently, a fifth embodiment will be described. FIG. 10 is a diagram illustrating a mobile X-ray apparatus 80. FIG. 11(a) is a schematic sectional view illustrating a state where a clutch 21a is engaged in the vicinity of the driving wheel 15. FIG. 11(b) is a schematic view illustrating the clutch 21a in an engaged state. FIG. 11(c) is a schematic sectional view illustrating a state where the clutch 21a is disengaged in the vicinity of the driving wheel 15. FIG. 11(d) is a schematic view illustrating the clutch 21a in a disengaged state. The mobile X-ray apparatus 80 is different from the mobile X-ray apparatus 1 and the like in the point that the motor 19 is directly connected to the driving wheel 15 without having the propeller shaft or the like therebetween.

The motor 19 is directly connected to each of the driving wheels 15 on both sides. In other words, the motor shaft 29 of the motor 19 serves as an axle of the driving wheels 15. The driving wheels 15 and the motor shaft 29 are connected to each other via the clutch 21a. In addition, the motor shaft 29 is provided with the clutch operation unit 27.

As illustrated in FIGS. 11(a) and 11(b), in the clutch 21a, a portion corresponding to the flywheel (hereinafter, a flywheel corresponding portion 87) is provided on the driving wheel 15 side, and a portion corresponding to the clutch plate (a clutch plate corresponding portion 85) is provided on the motor shaft 29 side on the inner side. The clutch plate corresponding portion 85 is divided into multiple pieces in the circumferential direction. The clutch plate corresponding portions 85 are normally pressed to the flywheel corresponding portion 87 on the outer circumferential side. Therefore, power is transmitted from the clutch plate corresponding portions 85 to the flywheel corresponding portion 87.

In the normal manipulation state, the above-described state is maintained at all times. Therefore, the driving wheels 15 rotate due to power from the motor shaft 29. Each driving wheel 15 is rotatably attached to the mobile cart 13 by a tubular support member 81, a bearing 83, and the like separately from the motor shaft 29. In other words, the motor shaft 29 is disposed inside the support member 81 which is fixed to the driving wheels 15. In addition, the support member 81 is rotatably supported by the bearing 83 provided in the mobile cart 13.

Meanwhile, in the manual manipulation state, the clutch operation unit 27 operates by the manipulation switch 43.

For example, as illustrated in FIGS. 11(c) and 11(d), when the manipulation switch 43 is pushed in, each of the divided clutch plate corresponding portions 85 returns to the center side (the arrow G directions in the diagram) . Therefore, the gap is generated between the clutch plate corresponding portions 85 and the flywheel corresponding portion 87, thereby generating slipperiness.

When the clutch plate corresponding portions 85 and the flywheel corresponding portion 87 are completely separated from each other, the driving wheels 15 rotate without being influenced by braking of the motor 19. Even in this case, the driving wheels 15 can rotate by being supported by the support members 81 in the mobile cart 13.

According to the fifth embodiment, it is possible to obtain an effect similar to that of the first embodiment. In addition, even in a case where the motor 19 and the driving wheels 15 are directly connected to each other, manipulation can be similarly performed. Such a mechanism can be provided between the motor 19 and the driving wheels via an excitation clutch. Torque of the motor 19 may be transmitted to the driving wheels 15 while the motor 19 and the battery 17 are electrically connected to each other. In a case of being electrically disconnected, the clutch may be disengaged.

Hereinbefore, with reference to the accompanying drawings, the embodiments of the present invention have been described. However, the technical scope of the present invention is not affected by the above-described embodiments.

It is clear that those skilled in the art can conceive of various types of changed examples and modification examples within the scope of the technical idea disclosed in Claims, and it is understood that the aforementioned examples naturally belong to the technical scope of the present invention as well.

For example, it is not necessary to mention that the configurations of the embodiments can be combined together.

### REFERENCE SIGNS LIST

1, 50, 60, 70, 80 MOBILE X-RAY APPARATUS; 3 X-RAY DEVICE; 5 X-RAY GENERATION UNIT; 7 X-RAY MOVABLE COLLIMATOR; ARM; 11 STRUT; 13 MOBILE CART; 15 DRIVING WHEEL; 17 BATTERY; 19 MOTOR; 21, 21a CLUTCH; 23 CART MANIPULATION UNIT; 25 SELECTOR SWITCH; 27 CLUTCH OPERATION UNIT; 29 MOTOR SHAFT; 31 PROPELLER SHAFT; 33 GEAR; 35 GEAR; 37 SHAFT; 41 MANIPULATING AMOUNT DETECTION UNIT; 43 MANIPULATION SWITCH; 45 SPACER; 51 VOLTAGE DETECTION UNIT; SWITCH OPERATION UNIT; 61 STORAGE UNIT; 71 ALARM UNIT; SUPPORT MEMBER; 83 BEARING; 85 CLUTCH CORRESPONDING PORTION; 87 FLYWHEEL CORRESPONDING PORTION

1. A mobile X-ray apparatus comprising:
a mobile cart;
an X-ray device that is mounted in the mobile cart and irradiates an object with an X-ray;
a motor that drives a driving wheel of the mobile cart via a clutch;
a battery that supplies electric power to the motor;
a cart manipulation unit that manipulates movement of the mobile cart; and
a selector switch that switches between two manipulation states which are a normal manipulation state and a manual manipulation state of the mobile cart,
wherein the motor is configured to brake when electric power stops being supplied from the battery,
wherein in a case where the normal manipulation state is selected by the selector switch, supplying of electric power to the motor is controlled in response to a manipulation of the cart manipulation unit,
wherein in a case where the manual manipulation state is selected by the selector switch, electric power stops being supplied from the battery to the motor, and the clutch is manipulated via a manipulation transmission mechanism in response to a manipulation of the cart manipulation unit, and
wherein the driving wheel and the motor are controlled so as to be attached to and be detached from each other in response to a manipulation of the clutch.

2. The mobile X-ray apparatus according to claim 1,
wherein the manipulation transmission mechanism includes a manipulation switch, a spacer which can perform an inserting and pulling-out operation between the manipulation switch and the cart manipulation unit, and a clutch operation unit which operates the clutch in accordance with a push-in amount when the manipulation switch is pushed in, and
wherein the manipulation switch is configured to be pushed in via the spacer which is inserted between the cart manipulation unit and the manipulation switch, in response to a manipulation of the cart manipulation unit.

3. The mobile X-ray apparatus according to claim 2,
wherein the manipulation transmission mechanism is configured to cause a maximum manipulating amount in a case where the cart manipulation unit is manipulated when the spacer is inserted to be substantially equal to a maximum manipulating amount in a case where the cart manipulation unit is manipulated when the spacer is not inserted.

4. The mobile X-ray apparatus according to claim 1, further comprising:
a voltage detection unit that detects a residual quantity of an electric capacity of the battery; and
a switch operation unit that operates the selector switch,
wherein when the residual quantity of the electric capacity detected by the voltage detection unit becomes equal to or less than a predetermined value, the switch operation unit operates the selector switch so as to switch over to the manual manipulation state from the normal manipulation state.

5. The mobile X-ray apparatus according to claim 4,
wherein the battery also supplies electric power to the X-ray device and further includes a storage unit which stores an estimated number of usages of the X-ray device set in advance, and
wherein the switch operation unit operates the selector switch so as to cause the residual quantity of the electric capacity detected by the voltage detection unit to ensure at least an electric capacity allowing as many times of irradiation with an X-ray as the estimated number of usages.

6. The mobile X-ray apparatus according to claim 4, wherein in a case where it is determined that the residual quantity of the electric capacity detected by the voltage
detection unit while the mobile cart is moving in the normal manipulation state becomes equal to or less than a predetermined value, the switch operation unit stops the motor and operates the selector switch after the mobile cart stops.

7. The mobile X-ray apparatus according to claim 4, further comprising:
an alarm unit that issues a warning to a user in a case where the voltage detection unit determines that the residual quantity of the electric capacity of the battery is equal to or less than a warning residual quantity,
wherein when the residual quantity of the electric capacity detected by the voltage detection unit becomes equal to or less than a predetermined value which is smaller than the warning residual quantity, the switch operation unit operates the selector switch.
